(19) United States
(12) Patent Application Publication
(10) Pub. No.: US 2010/0041877 A1
(43) Pub. Date: Feb. 18, 2010

(54) METHOD FOR PRODUCING PURIFIED ANTHOCYANIN

(75) Inventor: Hirotoshi Tamura, Kagawa (JP)

Correspondence Address: HAMRE, SCHUMANN, MUELLER & LARSON, P.C., P.O. BOX 2902, MINNEAPOLIS, MN, 55402-0902, US

(73) Assignee: National University Corporation Kagawa Univesity, Takamatsu-shi (JP)

(21) Appl. No.: 12/530,869

(22) PCT Filed: Mar. 11, 2008
(86) PCT No.: PCT/JP2008/054411

§ 371 Date: Sep. 11, 2009

(30) Foreign Application Priority Data
Mar. 12, 2007 (JP) 2007-062641

(51) Int. Cl. C07H 1/00; C07D 311/00

(52) U.S. Cl. 536/1.11; 549/398

(57) ABSTRACT

The present invention provides a method for producing a purified anthocyanin easily from a crude pigment fraction containing contaminants and the anthocyanin. A flavone that forms a metal complex with the anthocyanin and a metal ion of at least one of an alkaline-earth metal and a heavy metal is provided. Then, the flavonoid is brought into contact with the crude pigment fraction in a liquid in the presence of the metal ion of at least one of the alkaline-earth metal and the heavy metal, thereby forming a metal complex containing the anthocyanin, the flavonoid, and the metal ion. From the liquid, the contaminants contained in the crude pigment fraction are removed and the metal complex is collected. By subjecting this metal complex to, for example, an acid treatment, the anthocyanin is dissociatied from the metal complex. Thus, a high-purity anthocyanin is obtained.

### TECHNICAL FIELD

The present invention relates to a method for producing a purified anthocyanin from a crude pigment fraction containing contaminants and the anthocyanin.

### BACKGROUND ART

With the advance of petrochemistry, inexpensive synthetic pigments (tar colors) having excellent color representation and durability have been developed as colorants and used widely. However, although synthetic pigments are excellent in the above-described properties, there remains some doubt about their safety. Indeed, as a result of reexamination on the toxicity of synthetic pigments, only limited kinds of them are allowed to be used today. Lately, issues concerning food and environment have been rising, which leads to increased emphasis on safety. Thus, attention is being given again to pigments derived from natural products.

As the pigments derived from natural products, anthocyanins have been long known. Anthocyanins are pigments whose aglycons are anthocyanidins. For instance, they are useful as food additives, pigments for color inks etc., and colorants for pharmaceuticals etc. Moreover, since they are one kind of polyphenol, the effect as functional pigments also is expected. Such anthocyanins are present widely in, for example, flowers, leaves, fruit skins, fruits, etc. of plants, and natural colorants such as red cabbage pigments, safflower pigments, cochineal pigments, gardenia pigments, and grape skin pigments, for example, are in practical use. However, these natural colorants are not purified anthocyanins but are unpurified products with contaminants. Therefore, the provision of high-purity anthocyanins derived from natural products has been desired.

However, when purifying an anthocyanin from a natural product, various contaminants are contained in the natural product. Some of them may have, for example, a molecular weight or electric charge comparable with those of the anthocyanin. Thus, in order to obtain a high-purity anthocyanin, there is no other choice but to purify the anthocyanin by, for example, HPLC (high precision liquid chromatography). However, such a method is very cumbersome, and large-scale purification with this method is not practical. Moreover, anthocyanins are unstable in a weakly acidic aqueous solution, which makes the purification thereof very difficult (Non-Patent Document 1). Thus, although purified anthocyanins derived from natural products have excellent safety, they are very expensive and not readily available. For example, the cost of shisonin, which is contained in leaves of red perilla (Perilla frutescens var. crispa f. purpurea), is on the order of several tens of thousands yen per gram, so that it cannot be purchased easily.

### Non-Patent Document 1:

Goto, T., Kondo, T., Tamura, H., Imagawa, H., Iino, A. and Takeda, K.:

Structure of Gentiodelphin, an acylated anthocyanin isolated from Gentiana makinoi, that is stable in dilute aqueous solution, Tetrahedron Letters 23 (36), 3695-3698 (1982).

### Non-Patent Document 2:

Tadao Kondo, Kumi Yoshida, Atsushi Nagasawa, Takatoshi Kawai, Hirotoshi Tamura & Toshio Goto; Nature, Vol. 358, No 6386, (1992).

### Non-Patent Document 3:

Kondo, T., Ueda, M., Tamura, H., Yoshida, K., Isobe, M., and Goto, T.: Composition of Protocyanin, a self-assembled supramolecular pigment, from blue cornflower of Centaurea cyanus, Angewandte Chemie, Int. Ed English 33(9), 978-979 (1994).

### Non-Patent Document 4:

Yoshida, K., Kitahara, S., Ito, D., and Kondo, T., Phytochemistry, 67, 992 (2006).

### Non-Patent Document 5:

Takeda, K., Yanagisawa, M., Kifune, T., Kinoshita, T., Timberlake, C. F., Phytochemistry 35(5) 1167-1169, (1994).

### DISCLOSURE OF INVENTION

With the foregoing in mind, it is an object of the present invention to provide a method that allows a purified anthocyanin to be produced easily from a crude pigment fraction containing contaminants and the anthocyanin. In order to achieve the above object, a method for producing a purified anthocyanin according to the present invention is a method for producing a purified anthocyanin from a crude pigment fraction containing the anthocyanin, including the steps of:

(A) providing a flavonoid that forms a metal complex with the anthocyanin and a metal ion of at least one of an alkaline-earth metal and a heavy metal, the flavonoid being a flavonoid derived from a plant;
(B) bringing the flavonoid into contact with the crude pigment fraction in a liquid in the presence of the metal ion of at least one of the alkaline-earth metal and the heavy metal, thereby forming a metal complex containing the anthocyanin, the flavonoid, and the metal ion;
(C) collecting the metal complex from the liquid; and
(D) dissociating the anthocyanin from the metal complex.

Furthermore, a purified anthocyanin according to the present invention is a purified anthocyanin obtained by the production method of the present invention.

It has been reported that, among plants, Asiatic dayflowers (Commelina communis), for instance, contain, as a pigment, a metal complex (e.g., commelinin) formed of a delphinidin-type anthocyanin, a flavonoid (e.g., flavocommelin), and a metal ion (e.g., magnesium ion) (Non-Patent Document 2). It also has been reported that, a cyanidin-type anthocyanin, a flavonoid, and a metal ion forms a metal complex in a cornflower_(Centaurea cyanus), (Non-Patent Document 3), and similar metal complexes are formed in Himalayan blue poppies (Meconopsis hetonicifolia) and blue salvias (Salvia farinacea) (Non-Patent Documents 4 and 5). Thus, utilizing the property of anthocyanins to form a metal complex with a flavonoid and a metal ion spontaneously, the inventors of the present invention attempted to establish a method for purifying an anthocyanin from a crude pigment fraction containing contaminants. The metal complexes reported in the above-described papers are either a metal complex formed in a plant or a metal complex obtained by purifying a specific anthocyanin and a specific flavonoid as components of a metal complex independently and then reconstructing the metal complex using these independently-purified products in the presence of a metal ion. That is, in the technical field to which the present invention pertains, although the fact that an anthocyanin, a flavonoid derived from a plant, and a metal ion forms a metal complex has been reported, it has completely unknown that: even under the conditions where contaminants are present, a metal complex specifically incorporating an anthocyanin can be formed; a metal complex is formed when a plurality of kinds of anthocyanins are present; and furthermore, a metal complex is formed with a flavonoid and an anthocyanin in combinations other than those described in the papers. With the foregoing in mind, the inventors of the present invention conducted a keen study, and as a result, they found that, even when contaminants are present in a crude pigment fraction containing an anthocyanin, it is possible to form a metal complex by bringing a flavonoid derived from a plant as described above into contact with the anthocyanin in the crude pigment fraction in the presence of a metal ion. The inventors of the present invention further found that, from a metal complex containing an anthocyanin, contaminants contained therein together with the anthocyanin can be separated therefrom. In a crude pigment fraction containing an anthocyanin and contaminants, separating them is very difficult. However, from a metal complex containing an anthocyanin, contaminants contained therein together with the anthocyanin can be removed easily, which is the fact newly discovered by the inventors of the present invention. Furthermore, the anthocyanin can be dissociated easily from the metal complex containing the anthocyanin, which allows the purified anthocyanin to be prepared easily. Besides, the metal complex is of high purity because the contaminants have been removed therefrom, so that the purified anthocyanin obtained by dissociation also has a high purity.

Therefore, according to the present invention, it is possible to purify an anthocyanin from the crude pigment fraction easily in a simple manner, thus allowing a high-purity purified anthocyanin to be provided at low cost. Hence, it can be said that the present invention is very useful in all the fields where natural pigments are used, including, for example, a food field.

### BRIEF DESCRIPTION OF DRAWINGS

FIG. 1 is a chromatogram of a crude pigment fraction obtained by HPLC.

FIG. 2 is a chromatogram of a composite (metal complex) obtained by HPLC in an example of the present invention.

FIG. 3 is a graph plotting a molar ratio (amount-of-substance ratio) of Mg²⁺ to anthocyanins and a before-and-after ratio of malonylshisonin in another example of the present invention.

### DESCRIPTION OF THE INVENTION

The method for producing a purified anthocyanin according to the present invention is, as described above, a method for producing a purified anthocyanin from a crude pigment fraction containing the anthocyanin, including the steps of:

(A) providing a flavonoid that forms a metal complex with the anthocyanin and a metal ion of at least one of an alkaline-earth metal and a heavy metal, the flavonoid being a flavonoid derived from a plant;
(B) bringing the flavonoid into contact with the crude pigment fraction in a liquid in the presence of the metal ion of at least one of the alkaline-earth metal and the heavy metal, thereby forming a metal complex containing the anthocyanin, the flavonoid, and the metal ion;
(C) collecting the metal complex from the liquid; and
(D) dissociating the anthocyanin from the metal complex.

In the present invention, the metal complex containing the anthocyanin, the flavonoid derived from a plant, and the metal ion also is referred to as a “composite” hereinafter. It is to be noted that the production method of the present invention can also be referred to as a method for purifying an anthocyanin.

<Flavonoid Derived from a Plant>

Flavonoids derived from a plant to be used in the present invention can be those that form a metal complex with an anthocyanin and the above-described metal ion. Examples of such flavonoids include the following flavones (2-phenylchromones) and their derivatives. Examples of the derivatives include flavonols containing a hydroxyl group and derivatives containing a methoxy group. The flavonoids can be, for instance, flavones derived from plants. Specific examples thereof include: flavocommelin, which is an Asiatic dayflower-derived flavone (Non-Patent Document 2); apigenin 4′-(6-O-malonylglucoside)-7-glucuronide, which is a cornflower (Centaurea cyanus, Rodgersia)-derived flavone (Non-Patent Document 3); flavonol 3-getiobiose or flavonol 3-(6-O-glucosyl-b-O-galactoside), which is a Himalayan blue poppy-derived flavonol (Non-Patent Document 4); apigenin 7,4′-diglucoside, which is a blue salvia-derived flavone (Non-Patent Document 5); hesperidin and diosmin, which are citrus fruit-derived flavones; naringenin 7-glucoside, which is citrus fruit-derived flavone; rutin and apigenin, which are buckwheat-derived flavones; apigenin 7-glucoside, apigenin 7-o-neohesperidoside, and silymarin, which are flavones; and luteolin 7-glucoside, which is an olive-derived flavone.

These flavonoids can be prepared from plants, for instance. Generally, the above-described flavonoids are present in portions where anthocyanins are localized, so that they can be extracted from the portions of plants where anthocyanins are localized. An example of a method for preparing the above-described flavocommelin from Asiatic dayflowers will be described below as a specific example.

First, Asiatic dayflower petals are squeezed. Then, to the squeezed juice obtained, ethanol is added to carry out ethanol precipitation, and the supernatant is collect. The proportion of ethanol added is not particularly limited, but preferably is 4 to 20 times (in volume) the squeezed juice. The ethanol supernatant contains flavocommelin, and the precipitate obtained through the ethanol precipitation contains a metal complex (commelinin) composed of Asiatic dayflower-derived flavocommelin, Asiatic dayflower-derived anthocyanins (e.g., awobanin and malonylawobanin), and a metal ion. Subsequently, the supernatant is applied to an adsorbent, thereby adsorbing the flavocommelin to the adsorbent. The flavocommelin adsorbed is eluted by concentration gradient of methanol. At this time, considering the possibility that the flavocommelin contained in the supernatant may have formed a metal complex with the Asiatic dayflower-derived anthocyanins and the metal ion, it is preferable to supply an acidic aqueous solution to dissociate the Asiatic dayflower-derived anthocyanins from the adsorbed flavocommelin prior to the elution by the concentration gradient of methanol. As the adsorbent, “Amberlite XAD (trade name)” (synthetic adsorbent, manufactured by ORGANO CORPORATION.) can be used, for example. The acidic aqueous solution is not particularly limited, and can be, for example, an aqueous solution of hydrochloric acid or the like. A flavocommelin fraction eluted by the concentration gradient of methanol then is collected. This flavocommelin fraction may be used as it is. Alternatively, the flavocommelin fraction may be added to a mixed solution of an organic solvent and water after being concentrated, then crystallized through heating, and dried, and the thus-obtained product may be used, for example. Note here that the flavonoid is not limited as long as it can form a metal complex with an anthocyanin in a crude pigment fraction that will be described later. For example, a crude flavonoid fraction extracted from a plant may be used as it is.

Other flavonoids derived from a plant also can be prepared, for example, in the same manner as described above. For example, when preparing a target flavonoid from a Rodgersia or Himalayan blue poppy, it can be extracted from their petals, for example. As such flavonoids, synthetic compounds may be used, for example.

### <Metal Ion>

In the present invention, the metal ion may be a metal ion of at least one of an alkaline-earth metal and a heavy metal. Examples of the alkaline-earth metal ion include a magnesium ion. Examples of the heavy metal ion include ions of zinc, nickel, cadmium, iron, cobalt, aluminum, copper, manganese, chromium, and tin. Among these, magnesium is preferable.

In the present invention, the metal complex is formed in a liquid in the presence of a metal ion. Thus, it is preferable to add an ionizable metal compound to the liquid. Such a metal compound is not limited, and specific examples thereof include acetate, hydrochloride, sulfate, carbonate, and the like of alkaline-earth metals or heavy metals.

### <Crude Pigment Fraction>

The crude pigment fraction is not limited as long as it contains an anthocyanin. However, since the present invention is an anthocyanin purifying method particularly effective when contaminants are present with an anthocyanin, a crude pigment fraction containing contaminants is preferable. Examples of such a crude pigment fraction include plant extracts that have not been subject to a purifying treatment. The plant extract can be, for example, squeezed juice obtained from a portion of a plant where a target anthocyanin is contained. For example, when purifying an anthocyanin contained in petals, squeezed juice of the petals can be used; when purifying an anthocyanin contained in a fruit, squeezed juice of the fruit can be used; and when purifying an anthocyanin contained in a skin of a fruit or a seed, squeezed juice of the skin can be use. Furthermore, in the case where an anthocyanin cannot be extracted merely by squeezing, a portion of a plant where a target anthocyanin is contained may be squeezed after being immersed in a solvent, for example. The solvent is not limited, and can be, for example, water, an organic solvent such as methanol, and a mixed solvent thereof. The solvent further may contain trifluoroacetic acid or the like. Examples of the plant include perillas, red cabbages, grapes, black corns, red radishes, berries including strawberry, cassis, and blueberry, beans such as black bean and adzuki bean, potatoes such as purple sweet potato, rice such as red rice, red onions, olives, apples, and beans such as red kidney bean and black bean. Squeezed juice of such plants generally contains contaminants such as proteins, sugars, vitamins, minerals, lipids, flavonoids other than anthocyanins, and polymerized polyphenols.

From the crude pigment fraction thus extracted, polar components such as sugars and proteins may be removed in advance, for example, before forming a metal complex. This allows the anthocyanin to be purified from the crude pigment fraction more efficiently. The method for removing the polar components is not limited. This can be achieved, for example, by means of an adsorbent as described above, and specifically, “Amberlite XAD (trade name)” (synthetic adsorbent, manufactured by ORGANO CORPORATION.) or the like can be used. It should be noted that what poses a problem in the conventional art is the contaminants having a molecular weight or electric charge similar to those of anthocyanins (in particular, flavonoids other than anthocyanins, polymerized polyphenols, etc.), for example. Thus, removing sugars and proteins in advance, for example, in the preparation of the crude pigment fraction is not at all contrary to the effect of the present invention.

### <Anthocyanin>

In the present invention, the crude pigment fraction may contain one kind or two or more kinds of anthocyanins. Also, a purified anthocyanin fraction obtained finally by the production method of the present invention may contain one kind or two or more kinds of anthocyanins. Anthocyanins collectively refer to glycosides whose aglycons are anthocyanidins as described above, and there are a plurality of compounds called “anthocyanin”. In the conventional art, as described above, not separating these individual anthocyanins but, in the previous step, separating an anthocyanin and contaminants contained in a crude pigment fraction derived from a plant itself is difficult. In the present invention, the separation of contaminants and an anthocyanin can be carry out easily by forming a metal complex, and a purified anthocyanin fraction obtained finally may contain two or more kinds of anthocyanins. Moreover, even if there has been a technique for separating each individual anthocyanin in the conventional art, an anthocyanin fraction as a raw material thereof has a problem in its purity. However, according to the present invention, it is possible to provide a purified anthocyanin fraction with the contaminants contained therein being reduced, whereby such a problem can be solved. On this account, a purified anthocyanin fraction obtained by the present invention may contain two or more kinds of anthocyanins. Furthermore, it is well known in the art that not only a single anthocyanin derived from a natural product but also a mixture of a plurality of kinds of anthocyanins derived from a natural product has a sufficient commercial value.

As described above, although the kind of anthocyanin contained in a crude pigment fraction is not limited, the anthocyanin represented by, for example, the following formula is preferable. In the following formula, R¹ to R⁷ are not particularly limited, and each have, for example, a hydrogen atom, a hydroxyl group, or a functional group such as a methoxy group. The hydroxyl group can be replaced with a so-called glycoside with glucose or the like being bound thereto. The above-described functional groups may be identical to or different from each other, and at least one of them is a hydroxyl group. Furthermore, an anthocyanin having at least two hydroxyl groups on the B-ring of the anthocyanidin represented by the following formula is preferable because such an anthocyanin is more prone to form a metal complex with a flavonoid. That is, in the following formula, at least two of R¹, R², and R³ preferably are a hydroxyl group. Examples of such an anthocyanin include, but are not limited to, anthocyanins as peonidin-based glycosides, anthocyanins as delphinidin-based glycosides, anthocyanins as petunidin-based glycosides, and anthocyanins as delphinidin-based glycosides. Specific examples of the anthocyanin include malonylshisonin, methylmalonyl shisonin, shisonin, cyanine, malonylawobanin, awobanin, cyanidin, delphinidin, luteolinidin, petunidin, and europinidin. The term “anthocyanidin” and “anthocyanin” may refer to, in a limited sense, only specific ones among the compounds represented by the following formula and glycosides thereof. However, in the present invention, these terms have broader meanings and collectively refer to pigments present in, for example, flowers, leaves, fruit skins, fruits, and the like of plants.

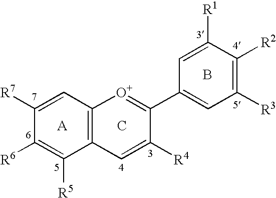

One example of anthocyanin will be given by the following formula.

TABLE 1

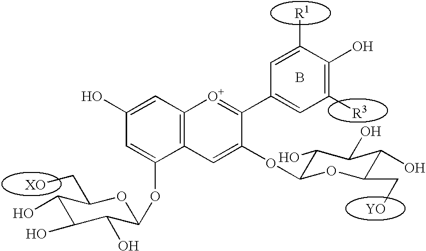

| | R¹ | R³ | X | Y |
|---|---|---|---|---|
| Malonylawobanin | OH | OH | Malonyl-H | p-coumaryl |
| Awobanin | OH | OH | H | p-coumaryl |
| Malonylshisonin | OH | H | Malonyl-H | p-coumaryl |
| (Methylmalonyl)shisonin | OH | H | Methylmalonyl | p-coumaryl |
| Shisonin | OH | H | H | p-coumaryl |
| Cyanin | OH | H | H | H |

Hereinafter, the production method of the present invention will be described by taking an example where Asiatic dayflower-derived flavocommelin is used as a flavonoid. It should be noted, however, the present invention is by no means limited to this example.

First, a crude pigment fraction extracted from a plant is provided in the above-described manner. The crude pigment fraction may be used as it is, but it is preferable to treat the crude pigment fraction with alkali before forming a metal complex. By such an alkali treatment, an anthocyanin contained in the crude pigment fraction turns to an anhydro base or an anhydro base anion. This allows a metal complex to be formed more efficiently. When converting an anthocyanin to an anhydro base, it is preferable to set the pH of the crude pigment fraction to, for instance, 5 to 9, more preferably 7 to 9. When converting an anthocyanin to an anhydro base anion, it is preferable to set the pH of the crude pigment preferably to, for instance, 9 or greater, more preferably 9 to 12. One example of the structural change of an anthocyanin is shown in the following formula.

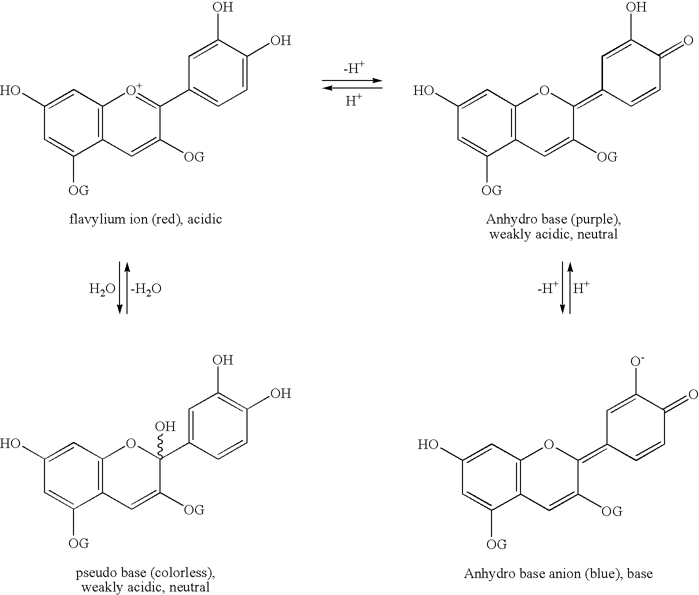

Next, in the manner as described above, flavocommelin is provided, and the flavocommelin, the crude pigment fraction, and a metal ion are added to a solvent and mixed. At this time, the order of adding the flavocommelin, the crude pigment fraction, and the metal ion is not limited. The solvent is not limited, and water (e.g., pure water) or the like can be used, for instance. In addition to this, the solvent may be a liquid extract from a plant containing an anthocyanin itself. The flavocommelin and the crude pigment fraction each may be solid or liquid. To provide the metal ion, for example, a metal compound that ionizes in the above-described liquids may be added to the solvent directly, or a metal compound solution containing a metal that already has been ionized (e.g., a magnesium acetate aqueous solution) may be added. The pH of the mixed solution containing the flavocommelin, the crude pigment fraction, and the metal ion preferably is 6 to 9, for example. There is no limitation on temperature conditions. However, the temperatures lower than 50° C., e.g., 20° C. to 30° C., are generally preferable because decomposition of flavonoids and anthocyanins can be avoided sufficiently. A metal complex composed of the flavocommelin, the anthocyanin, and the metal ion is formed instantaneously by mixing these components in the liquid.

The ratio of the respective components in the liquid can be decided as appropriate, and there is no limitation thereon. However, it is preferable to add sufficient amounts of the flavocommelin and the metal ion relative to the anthocyanin contained in the crude pigment fraction. In a metal complex commelinin formed in Asiatic dayflowers, an anthocyanin, a flavonoid (flavocommelin), and a metal ion generally are present at the molar ratio of 6:6:2 (Non-Patent Document 2). It is preferable that the flavocommelin is present in an amount of, for example, 1 to 20 mol, more preferably 1 to 5 mol, with respect to 1 mol of the anthocyanin in the crude pigment fraction. Furthermore, it is preferable that the metal ion is present in an amount of, for example, 10 to 50 mol, more preferably 10 to 20 mol, with respect to 1 mol of the anthocyanin in the crude pigment fraction. The ratio (A:F:M) of the anthocyanin (A), the flavocommelin (F), and the metal ion (M) preferably is, for example, (A) 1: (F) 1 to 5: (M) 5 to 20, more preferably (A) 1: (F) 1 to 2: (M) 5 to 20. In the case where another flavonoid derived from a plant is used, the same ratio can be applied.

Since the examples described later demonstrate that a metal complex can be formed by such a method, it is not essential to confirm whether or not the composite has been formed in the production method of the present invention. Whether or not the metal complex has been formed can be confirmed by the method described in Non-Patent Document 2, for example. More specifically, the presence of the metal complex can be determined by: the shift of an absorption spectrum in the visible region to a longer wavelength side; the fact that an exiton-type negative Cotton effect of CD is observed at 580 nm (θ; +615,000) and 668 nm (θ; −480,000); and elution of a macromolecule blue composite in molecular sieve column chromatography.

Next, the metal complex is collected from the liquid. Contaminants contained in the crude pigment fraction are thereby removed. The metal complex formed in the above-described steps is a composite composed of specific components (namely, the flavonoid, the anthocyanin, and the metal ion), and even when the crude pigment fraction contains various contaminants, there is little chance that these contaminants might form a metal complex with the flavocommelin and the metal ion. Thus, by collecting this metal complex, other components can be removed and the metal complex containing the anthocyanin can be collected with high purity. Once the metal complex containing the target anthocyanin is collected, dissociating the anthocyanin from the metal complex and separating the dissociated anthocyanin, the flavocommelin, and the metal ion from one another as will be described later can be achieved easily. Thus, the contaminants contained in the crude pigment fraction are removed with very high efficiency, thus allowing a high-purity purified anthocyanin to be obtained. Furthermore, the present invention is intended to remove contaminants in a crude pigment fraction. Thus, as described above, a purified anthocyanin fraction obtained finally may contain one kind or two or more kinds of anthocyanins.

The method for separating the metal complex and the contaminants from the liquid is not particularly limited, and can be an ethanol precipitation method, for example. As described above, commelinin in the Asiatic dayflower is contained in, for example, a precipitate fraction of ethanol precipitation. Therefore, by carrying out ethanol precipitation, the metal complex composed of the flavocommelin, the anthocyanin, and the metal ion in the present invention can be collected similarly from its precipitate fraction. The proportion of ethanol added is not limited. With respect to the liquid in which the composite is formed (hereinafter also referred to as “reaction solution”) in the above-described steps, the amount of ethanol is, for example, 3 to 10 times (in volume) the liquid, preferably 3 to 6 times (in volume) the liquid, although it can be determined as appropriate depending on the moisture content, for example.

In addition to the above-described method, it is possible to separate the metal complex and the contaminants by molecular weight fractionation such as gel filtration, for example. The gel filtration can be carried out by using, for example, gel filtration column chromatography, in which various types of conventionally known columns can be used. Specific examples of the column include “Sephadex (trade name)” (manufactured by Amersham).

By dissociating the anthocyanin from the thus-collected metal complex, it is possible to obtain a high-purity purified anthocyanin. The method for dissociating the anthocyanin is not limited, and can be, for example, subjecting the metal complex to an acid treatment, a heat treatment, or an ultrasonic treatment. The anthocyanin may be dissociated by treating the metal complex with a chelating agent such as EDTA so as to remove the metal ion therefrom. The dissociation of the anthocyanin will be explained below by taking an example where an acid treatment is employed among the above-described methods. After the metal complex has been subjected to the acid treatment, the flavocommelin remains electrically neutral while the anthocyanin is positively charged. Thus, when a cation-exchange resin (an acidic ion-exchange resin), for instance, is used, the positively charged anthocyanin is adsorbed to the ion-exchange resin while the neutral flavonoid is not adsorbed thereto. After separating the unadsorbed flavonoid, an acid is brought into contact with the cation-exchange resin to which the anthocyanin has been adsorbed. The anthocyanin is thereby eluted, resulting in dissociation of the anthocyanin from the metal complex. In this manner, the contaminants contained in the crude pigment fraction can be removed, thus allowing the high-purity purified anthocyanin to be collected. According to such a method, the anthocyanin can be dissociated merely by conducting the acid treatment, so that the purified anthocyanin can be prepared easily.

The flavocommelin, the anthocyanin, and the metal ion after being subjected to the acid treatment can be separated from one another in the following manner, for example.

As described above, as a result of the acid treatment to dissociate the anthocyanin from the metal complex, the flavocommelin maintains an electrically neutral state while the anthocyanin is brought to a positively charged state. Thus, by applying a mixed solution containing them and the metal ion to, for example, the cation-exchange resin, it becomes possible to collect only the anthocyanin. That is, when the mixed solution is applied to the cation-exchange resin, the positively charged anthocyanin is adsorbed to the cation-exchange resin while the electrically neutral flavocommelin is not adsorbed thereto. Thus, after removing the unadsorbed flavocommelin, the adsorbed anthocyanin is eluted by, for instance, the concentration gradient of a salt, and the elution fraction is collected. In this manner, the flavocommelin and the anthocyanin further can be separated from each other.

### EXAMPLES

Hereinafter, the present invention will be described by way of examples with reference to comparative examples. It is to be noted, however, the present invention is by no means limited to the following examples.

### Example 1

### (1) Extraction of Crude Pigment from Perilla

2600 g of red perilla leaves were pulverized with a mixer and then immersed in 8.85 L of 50% MeOH containing 0.5% TFA (trifluoroacetic acid) at 4° C. overnight. Next, the immersed leaves were taken out and then squeezed with a hand-screw squeezing machine with the use of 2.55 L of 50% MeOH containing 0.5% TFA. The liquid extract was then collected. A residue obtained after the squeeze again was immersed in 3 L of 50% MeOH containing 0.5% TFA at 4° C. overnight. It then was subjected to the same compressing and extracting treatment. The liquid extract obtained was mixed with the previously-obtained liquid extract (the resultant mixture was about 15 L). Then, using an evaporator and a vacuum pump, the mixture was concentrated to dryness under reduced pressure (115.9 g). 49.58 g of the thus-obtained dried solid was dissolved in a MeOH aqueous solution containing 1% HCl. The solution was filtered, after which the pigment in the solution was adsorbed to an ion-exchange column (trade name: “Amberlite” (registered trademark) XAD-7”, column: Φ25 mm×450 mm). Then, in order to remove polar components such as sugars and proteins, 1 L of a 0.5% TFA aqueous solution was caused to flow through the column. Subsequently, 1.5 L of an 80% MeOH aqueous solution containing 0.5% TFA further was caused to flow through the column, thereby eluting the adsorbed pigment. The collected eluate was concentrated to dryness under reduced pressure using an evaporator. Then, in order to remove a trace amount of moisture and the TFA as a volatile acid, it was further dried out using a vacuum pump. Thus, 12.3 g of a crude pigment was obtained.

### (2) Isolation of Flavocommelin from Asiatic Dayflower

### (2-1) Isolation of Crude Commelinin

4.4 kg of frozen blue petals of Asiatic dayflowers were squeezed with a hand-screw squeezing machine, whereby 3.64 L of blue squeezed juice was obtained. 21 L of ethanol was added thereto, and the resultant mixture was left to stand at −20° C. overnight to settle a pigment fraction. This was separated into a supernatant and a precipitate by centrifugation (SCR 20B (trade name), manufactured by HITACHI, 5000 ppm, 10 minutes), and the precipitate was collected. The collected precipitate was dried over calcium chloride under reduced pressure. As a result, 14.5 g of crude commelinin in the form of a blue solid was obtained. Absorption spectrum data of the crude commelinin and the measurement conditions thereof are shown below.

*UV spectrum measurement conditions

Device: JASCO Corporation
trade name “V-520-SR type spectrophotometer”

Solvent: 0.05 M acetate buffer (pH 5.6)

Cell length: 1 mm

*Absorption spectrum data of Commelinin

UV-vis λ nm (ε): 645 (66000), 590 (140200)

### (2-2) Isolation and Purification of Flavocommelin

The supernatant separated after the ethanol precipitation in the item (2-1) above was concentrated to dryness under reduced pressure using an evaporator and a vacuum pump. Thus, dark blue oil containing a large amount of flavocommelin and a yellow solid were obtained. The yellow solid was dissolved in dimethyl sulfoxide, and the resultant solution was analyzed by HPLC. As a result, the yellow solid was identified as “flavocommelin”, which is one kind of flavone. This solution was mixed with the dark blue oil diluted with ultrapure water, and the resultant mixture was filtered. Thereafter, the flavocommelin contained in the solution was adsorbed to an ion-exchange column chromatography (trade name: “Amberlite” (registered trademark) XAD-7, column: φ25 mm×450 mm). First, 0.72 L of a 1% hydrochloric acid aqueous solution was caused to flow through the column. By this process, even in the case where the flavocommelin adsorbed to the ion-exchange resin forms a composite with an Asiatic dayflower-derived anthocyanin, the Asiatic dayflower-derived anthocyanin can be dissociated from the flavocommelin. Subsequently, 7.5 L of ultrapure water, 6 L of a 20% MeOH aqueous solution, 6 L of a 30% MeOH aqueous solution, 9 L of a 40% MeOH aqueous solution, and 3 L of a 60% MeOH aqueous solution were caused to flow through the column in this order, thereby eluting the adsorbed flavocommelin. Elution of the flavocommelin was confirmed from the time point when 2 L of the 20% MeOH aqueous solution had flowed through the column. Thus, the flavocommelin having been eluted from this time point to the time point when 5 L of the 40% MeOH aqueous solution had flowed through the column was collected as a fraction I; the flavocommelin having been eluted from the time point when 5 L of the 40% MeOH aqueous solution had flowed through the column to the time point when 1 L of the 60% MeOH aqueous solution had flowed through the column was collected as a fraction II; and the flavocommelin having been eluted from the time point when 1 L of the 60% MeOH aqueous solution had flowed through the column to the time point when 3 L of the 60% MeOH aqueous solution had flowed through the column was collected as a fraction III. The fraction I was concentrated under reduced pressure, whereby 6.98 g of a dried solid was obtained. From the fractions II and III, no dried solid was obtained. To the dried solid obtained from the fraction I, 50 mL of ultrapure water and 100 mL of acetonitrile were added. The resultant mixture was heated, whereby the flavocommelin was precipitated. The precipitated flavocommelin was dried under reduced pressure, whereby 5.28 g of the flavocommelin (purity: 90%) was obtained. Absorption spectrum data of the thus-obtained flavocommelin and the measurement conditions thereof are shown below. The presence of commelinin can be determined by, as shown in the Non-Patent Document 2, the shift of an absorption spectrum in the visible region to a longer wavelength side and a strong exiton-type negative Cotton effect in the visible region.

### *TV Spectrum Measurement Conditions

Device: JASCO Corporation,
trade name “V-520-SR type spectrophotometer”

Solvent: ultrapure water

Cell length: 1 mm

### *Absorption Spectrum Data of Flavocommelin

UV-vis λ nm (E): 326 (19800), 271 (22260)

(2-3) Isolation of Crude Pigment and Flavocommelin from Asiatic Dayflower Residue

The residue obtained after squeeze in the item (2-1) above was immersed in 3 L of 50% MeOH containing 0.5% TFA at 4° C. for one month, after which it was separated into a liquid extract and a residue. This residue was immersed again in 3 L of 50% MeOH containing 0.5% TFA. Then, extraction was carried out in the same manner as in the above, and the liquid extract was collected. This liquid extract was mixed with the previously-obtained liquid extract. Then, using an evaporator and a vacuum pump, the resultant mixture was concentrated to dryness under reduced pressure (10.18 g). 10.18 g of the thus-obtained dried solid was dissolved in ultrapure water. The solution then was filtered, after which a pigment fraction and flavocommelin in the solution were adsorbed to an ion-exchange column chromatography (trade name: “Amberlite” (registered trademark) XAD-7, column: (D25 mm×450 mm). Then, in order to remove polar components such as sugars and proteins, 4 L of a 0.5% TFA aqueous solution was caused to flow through the column. Subsequently, 2 L of a 10% MeOH aqueous solution, 4 L of a 30% MeOH aqueous solution, 2 L of a 40% MeOH aqueous solution further were caused to flow through the column in this order, thereby eluting the adsorbed flavocommelin. Elution of the flavocommelin was confirmed from the time point when the 30% MeOH aqueous solution had flowed through the column. Thus, an eluate was collected from this time point. The collected fraction was concentrated under reduced pressure, whereby a dried solid was obtained. To this dried solid, 20 mL of ultrapure water and 150 mL of acetonitrile were added. The resultant mixture was heated, whereby the flavocommelin was precipitated. The precipitated flavocommelin was dried under reduced pressure, whereby 0.61 g of the flavocommelin was obtained. Subsequently, after the elution of the flavocommelin, 1.5 L of an 80% MeOH aqueous solution containing 0.5% TFA was caused to flow through the ion-exchange column, thereby eluting the adsorbed pigment fraction. The collected eluate was concentrated to dryness under reduced pressure using an evaporator. Then, in order to remove a trace amount of moisture and the TFA as a volatile acid, it was further dried out with a vacuum pump. Thus, 0.82 g of a crude pigment of the Asiatic dayflower was obtained.

### (3) Formation of Composite of Perilla-Derived Anthocyanin and Asiatic Dayflower-Derived Flavocommelin

To 3 mg of the perilla-derived crude pigment (dried solid) prepared in the item (1) above, 1 mL of ultrapure water was added. The mixture was neutralized with 1 mL of 0.5 N ammonia water, after which it was concentrated to dryness under reduced pressure. Through this neutralization, the anthocyanin contained in the perilla-derived crude pigment was transformed to an anhydro base form. To this dried solid, 50 μL of ultrapure water and a flavocommelin solution obtained by dissolving 3 mg of the flavocommelin prepared in the above item (2-2) in 50 μL of water were added. Further, 25 μL of a 0.5 M magnesium acetate aqueous solution was added thereto. This mixed solution turned blue instantaneously upon mixing. The mixed solution was concentrated under reduced pressure. The composite in the mixed solution was adsorbed to a gel filtration column chromatography (trade name: Sephadex G-10, column: Φ10 mm×150 mm) and purified by being eluted with water. Then, the deep blue elution fraction was collected as a composite-containing fraction. The formation of the composite containing the perilla derived-anthocyanin and the Asiatic dayflower-derived flavocommelin was confirmed, similarly to the case of commelinin described above, by the shift of an absorption spectrum in the visible region to a longer wavelength side and a strong exiton-type negative Cotton effect in the visible region.

The yield of the composite of the perilla-derived anthocyanin and the Asiatic dayflower-derived flavocommelin was determined. First, the crude pigment used in the formation of the composite was dissolved in MeOH containing 1% TFA, and the absorbance measurement was carried out. On the other hand, to the formed composite, MeOH containing 1% TFA was added so as to achieve the same dilution factor to that of the crude pigment. This solution was then subjected to an ultrasonic treatment for 10 minutes, thereby dissociating the perilla-derived anthocyanin from the composite. Thereafter, the absorbance measurement was carried out. The absorbance (C) of the composite was divided by the absorbance (P) of the crude pigment, and a percentage of the obtained value (100×C/P) was regarded as the yield (%) of the composite. As a result, since the absorbance (P) of the crude pigment was 0.514 and the absorbance (C) of the composite was 0.144, the yield (100×C/P) was found to be “about 28.02%”.

### *UV Spectrum Measurement Conditions

Device: JASCO Corporation,
trade name “V-520-SR type spectrophotometer”

Solvent: MeOH containing 1% TFA

Cell length: 1 mm

Wavelength: 529 nm (crude pigment), 529 nm (composite)

As described above, the fact that the formation of the composite was confirmed indicates that, when using a crude pigment containing contaminants, the composite of an anthocyanin and flavocommelin can be formed, and that the anthocyanin can be purified by dissociating the anthocyanin. Furthermore, it is known that a leaf of red perilla contains at least four kinds of anthocyanins. It was also found that, when using a crude pigment containing two or more kinds of anthocyanins as in the above, the composite of an anthocyanin and flavocommelin can be formed. These results demonstrate that, according to the present invention, when using, for instance, leaves of red perilla as a raw material, it is possible to purify anthocyanins by merely forming composites (metal complexes) thereof.

The composite of each kind of perilla-derived anthocyanin and the Asiatic dayflower-derived flavocommelin was blue that was more purplish than the composite of the Asiatic dayflower-derived anthocyanin and the Asiatic dayflower-derived flavocommelin. Conceivably, this is because the maximum absorption wavelength in the visible region of the former composite shifted to a shorter wavelength side as compared to that of the latter composite. The cause thereof presumably is that the nuclei of the perilla-derived anthocyanins are cyanidin while the nucleus of the Asiatic dayflower-derived anthocyanin, which is inherently contained in the Asiatic dayflower-derived flavocommelin, is delphinidin.

### (4) Analysis of Components of Composite

Next, whether or not the flavocommelin had formed a composite with each kind of anthocyanin was checked. As described above, a leaf of red perilla contains at least shisonin (S), malonylshisonin (MS), (methylmalonyl)shisonin (MMS) and cyanine (C). Thus, the compositional ratio at which each kind of anthocyanin and the flavocommelin formed the composite was determined.

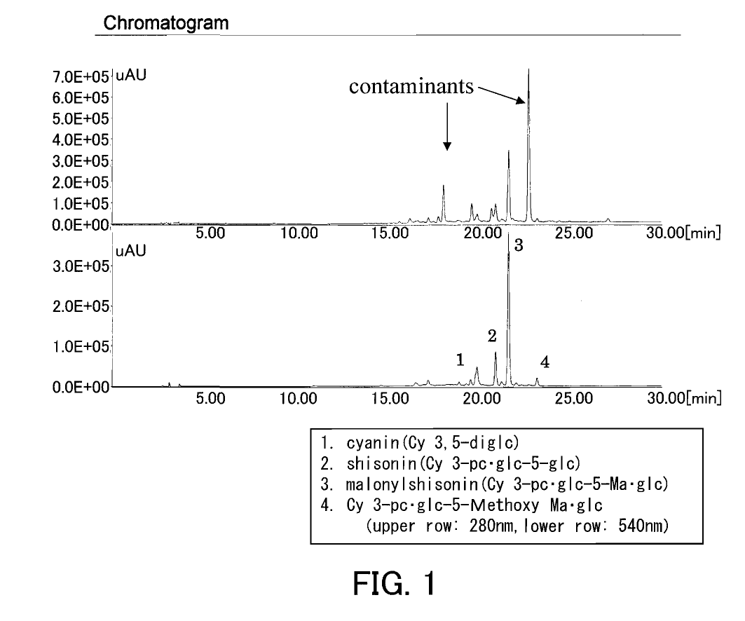

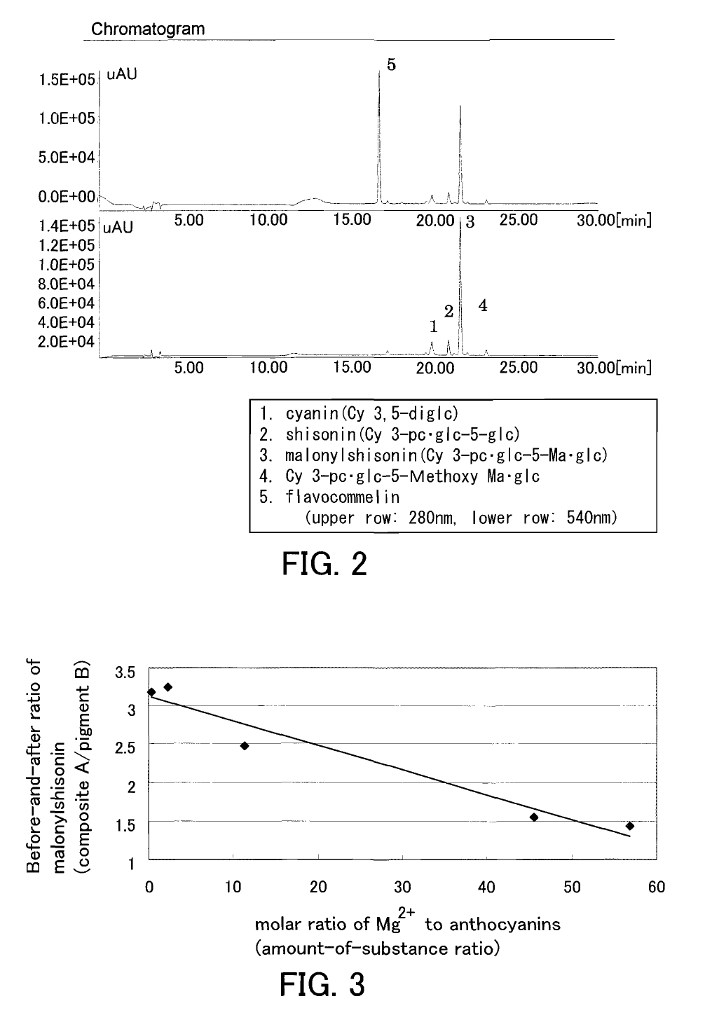

The crude pigment used in the item (3) and the composite formed in the item (3) were analyzed by HPLC carried out under the following conditions. The results are shown in FIGS. 1 and 2. FIG. 1 shows a HPLC chromatogram of the crude pigment, and FIG. 2 shows a HPLC chromatogram of the composite. As shown in the HPLC conditions below, mobile phases A and B are acidic solvents. Thus, by applying the composite to the HPLC, the anthocyanin was dissociated from the composite. Therefore, peaks in FIG. 2 directly indicate peaks of the respective kinds of anthocyanins having been dissociated, but they indirectly indicate peaks of the composites containing the respective anthocyanins. That is, from the peaks in FIG. 2, the amount of the composite incorporating each of the anthocyanins, the ratio of the respective composites, etc. can be determined (hereinafter the same).

As shown in the upper row of FIG. 1, in the chromatogram of the crude pigment, peaks of contaminants (e.g., proteins and organic acids, flavonoids other than anthocyanins, and polymerized polyphenols) are observed in the ultraviolet region (280 nm). In contrast, as shown in the upper row of FIG. 2, in the chromatogram of the composite, only peaks of flavocommelin and a trace amount of impurities were detected. From this, it can be seen that most of the contaminants contained in the crude pigment, in particular, flavonoids other than anthocyanins and polymerized polyphenols conventionally causing problems, are substances not suitable for forming composites, and thus they did not participate in the formation of the composites and removed by the above-described column chromatography.

*Conditions of HPLC analysis

| | |
|---|---|
| Fluid pump: | JASCO Corporation, trade name “PU-980” × 2 |
| Detector: | JASCO Corporation, |
| | trade name “MD2010 Plus” photodiode |
| | array detector |
| Column: | trade name “Develosil ODS-HG-5” 250 mm × |
| | 4.6 mm |
| Mobile phase A: | acetic acid:acetonitrile:water:phosphoric acid = |
| | 2:2.5:94:1.5 |
| Mobile phase B: | acetic acid:acetonitrile:water:phosphoric acid = |
| | 20:25:53.5:1.5 |
| Amount of sample | 10 μL |
| injected: | |
| Flow rate: | 1 mL/min |
| Column temperature: | 40° C. |

TABLE 2
*Gradient time program

| | mobile | |
|---|---|---|
| time (min.) | phase A | mobile phase B |
| 0 | 100 | 0 |
| 5 | 100 | 0 |
| 25 | 0 | 100 |
| 30 | 0 | 100 |
| 31 | 100 | 0 |

Then, with regard to the crude pigment, based on the chromatogram at 540 nm, the proportion (%) of the peak area of each of the anthocyanins (S, MS, MMS and C) was determined. This is regarded as the compositional proportion (%) of each anthocyanin in the crude pigment. Furthermore, assuming that the proportion (%) of the peak area of cyanine (S) was 1, the ratios of the peak areas of other anthocyanins were determined. On the other hand, with regard to the composite formed in the item (3) above, based on the chromatogram at 540 nm, the proportion (%) of the peak area of each anthocyanin contained in the corresponding composite was determined. This is regarded as the compositional proportion (%) of the composite containing the corresponding anthocyanin. Furthermore, with regard to the composite, assuming that the proportion (%) of the peak area of S was 1, the ratios of the peak areas of other anthocyanins were determined. The results are shown in the following table. The composite formation was carried out three times, and the average value and the standard deviation were determined.

TABLE 3

| | proportion (%) | peak area ratio |
|---|---|---|
| <crude pigment> | | |
| S | 15.24 ± 0.08 | 1 |
| MS | 72.64 ± 0.57 | 4.77 ± 0.06 |
| MMS | 3.88 ± 0.11 | 0.26 ± 0.01 |
| C | 8.24 ± 0.60 | 0.54 ± 0.04 |
| <composite> | | |
| S | 15.59 ± 0.11 | 1 |
| MS | 70.57 ± 0.64 | 4.63 ± 0.25 |
| MMS | 5.37 ± 0.64 | 0.35 ± 0.06 |
| C | 8.80 ± 0.58 | 8.80 ± 0.58 |

As shown in Table 3 above, it was found that the crude pigment contained anthocyanins S, MS, MMS, and C as main components in the above ratio. With regard to the composite, it was found that the composites containing the anthocyanins S, MS, MMS, and C, respectively, were formed in the above ratio. These results demonstrate that, even when a plurality of kinds of anthocyanins are present as in the case of the above-described crude pigment, composites of anthocyanins and flavocommelin can be formed, and that flavocommelin can form a composite with various kinds of anthocyanins. It should be noted that, although the liquid extract from red perilla leaves was treated with Amberlite in order to remove polar components such as proteins from the liquid extract in the present example, it has been confirmed that composites were formed similarly when a crude pigment not subjected to this treatment was used and anthocyanins could be purified by the formation of the composites.

### Example 2

As shown in Example 1, it was found that, even in the case where a plurality of kinds of anthocyanins were present, it was possible to form composites of the respective anthocyanins with flavocommelin. Now, the selectivity of each of the anthocyanins when forming a composite was checked by varying the compositional ratio of the anthocyanins in the crude pigment.

### (1) Preparation of Crude Pigment

### (1-1) Acid Hydrolysis of Crude Pigment

20.6 g of the perilla crude pigment prepared in Example 1 was dissolved in a MeOH aqueous solution containing 1% HCl. This was allowed to stand for two days at room temperature with a hydrolysis reaction being observed by HPLC, whereby malonic acid was removed from malonylshisonin contained in the crude pigment. Thus, malonylshisonin was transformed to shisonin. This reaction solution was concentrated to dryness under reduced pressure. Then, in order to remove a trace amount of moisture and the HCl as a volatile acid, it was dried out further with a vacuum pump. Thus, 13.45 g of a crude pigment containing shisonin as a main component was obtained. This is referred to as a crude pigment A. Furthermore, 2.0 g of the perilla crude pigment prepared in the above-described example was dissolved in a MeOH aqueous solution containing 3% HCl. This was allowed to stand for one day at room temperature with a hydrolysis reaction being observed by HPLC, whereby malonic acid was removed from malonylshisonin contained in the crude pigment. Thus, malonylshisonin was transformed to shisonin. This reaction solution was concentrated to dryness under reduced pressure. Then, in order to remove a trace amount of moisture and the HCl as a volatile acid, it was further dried out with a vacuum pump. Thus, 1.7 g of a crude pigment containing shisonin as a main component was obtained. This is referred to as a crude pigment B.

### (1-2) Adjustment of Blending of Crude Pigment Components

Each of the crude pigments A and B obtained by the acid hydrolysis treatment was mixed with the crude pigment prepared in Example 1. Thus, crude pigments with different compositions were prepared. The compositional ratio thereof was set so that the ratio of shisonin (S) and malonylshisonin (MS) (the amount-of-substance ratio S MS) would be a predetermined ratio (1:0.25, 1:0.4, 1:0.49, 1:2.42, 1:3.71, and 1:4.31). The compositional ratio was checked by conducting HPLC analysis and determining peak area ratios of MS and S.

### (2) Analysis of Components of Composite

Composites were formed in the same manner as described in the item (3) in Example 1, and purification was conducted by column chromatography. Then, HPLC analysis of composite-containing fractions was performed in the same manner as described in the item (4) in Example 1. Then, the proportion (%) of a peak area of each of the crude pigments with different compositions and the composites was determined. Further, assuming that the proportion (%) of the peak area of cyanine (S) was 1, the ratio of the peak area of each of the crude pigments and the composites was determined. Moreover, with regard to each kind of anthocyanin, the ratio of the peak area ratio (B) of the crude pigment before the composite formation and the peak area ratio (A) of the composite after the composite formation (the before-and-after ratio A/B) was determined. As the before-and-after ratio A/B becomes relatively greater, it means that the corresponding anthocyanin is incorporated in the composite more selectively. The formation of the composite was carried out three times, and the average value and the standard deviation were determined. These results are shown in the following tables.

TABLE 4
S:MS = 1:0.25

| | | | before-and-after |
|---|---|---|---|
| | proportion | | ratio |
| | (%) | peak area ratio | A/B |
| <crude pigment> | | | |
| S | 57.20 ± 0.10 | 1 | — |
| MS | 14.39 ± 0.01 | 0.25 ± 0.00 | — |
| MMS | 16.68 ± 0.10 | 0.29 ± 0.00 | — |
| C | 11.72 ± 0.01 | 0.20 ± 0.00 | — |
| <composite> | | | |
| S | 42.49 ± 1.19 | 1 | 1 |
| MS | 22.06 ± 1.40 | 0.52 ± 0.02 | 2.08 |
| MMS | 24.44 ± 2.57 | 0.57 ± 0.05 | 1.97 |
| C | 14.68 ± 1.58 | 0.35 ± 0.03 | 1.75 |

TABLE 5
S:MS = 1:0.4

| | | | before-and-after |
|---|---|---|---|
| | proportion | | ratio |
| | (%) | peak area ratio | A/B |
| <crude pigment> | | | |
| S | 51.22 ± 0.35 | 1 | — |
| MS | 20.22 ± 0.07 | 0.40 ± 0.00 | — |
| MMS | 18.27 ± 0.08 | 0.36 ± 0.00 | — |
| C | 10.46 ± 0.07 | 0.21 ± 0.00 | — |
| <composite> | | | |
| S | 35.43 ± 3.11 | 1 | 1 |
| MS | 31.74 ± 1.23 | 0.90 ± 0.05 | 2.25 |
| MMS | 20.43 ± 2.35 | 0.58 ± 0.04 | 1.61 |
| C | 12.39 ± 6.01 | 0.36 ± 0.19 | 1.71 |

TABLE 6
S:MS = 1:0.49

| | | | before-and-after |
|---|---|---|---|
| | proportion | | ratio |
| | (%) | peak area ratio | A/B |
| <crude pigment> | | | |
| S | 48.00 ± 0.28 | 1 | — |
| MS | 23.53 ± 0.09 | 0.49 ± 0.00 | — |
| MMS | 19.31 ± 0.39 | 0.41 ± 0.01 | — |
| C | 9.36 ± 0.16 | 0.19 ± 0.00 | — |
| <composite> | | | |
| S | 32.87 ± 5.54 | 1 | 1 |
| MS | 34.80 ± 1.63 | 1.08 ± 0.23 | 2.20 |
| MMS | 15.12 ± 0.91 | 0.47 ± 0.08 | 1.15 |
| C | 17.18 ± 4.66 | 0.55 ± 0.22 | 2.89 |

TABLE 7
S:MS = 1:2.42

| | | | before-and-after |
|---|---|---|---|
| | proportion | | ratio |
| | (%) | peak area ratio | A/B |
| <crude pigment> | | | |
| S | 23.98 ± 0.09 | 1 | — |
| MS | 58.12 ± 0.61 | 2.42 ± 0.03 | — |
| MMS | 7.73 ± 0.10 | 0.32 ± 0.00 | — |
| C | 10.17 ± 0.58 | 0.42 ± 0.02 | — |
| <composite> | | | |
| S | 17.33 ± 0.07 | 1 | 1 |
| MS | 63.55 ± 0.99 | 63.55 ± 0.99 | 1.52 |
| MMS | 7.97 ± 0.55 | 7.97 ± 0.55 | 1.44 |
| C | 11.15 ± 0.38 | 11.15 ± 0.38 | 1.52 |

TABLE 8
S:MS = 1:3.71

| | | | before-and-after |
|---|---|---|---|
| | proportion | | ratio |
| | (%) | peak area ratio | A/B |
| <crude pigment> | | | |
| S | 17.73 ± 0.02 | 1 | — |
| MS | 65.64 ± 0.03 | 3.71 ± 0.00 | — |
| MMS | 5.74 ± 0.03 | 0.32 ± 0.00 | — |
| C | 10.90 ± 0.02 | 0.62 ± 0.00 | — |
| <composite> | | | |
| S | 14.92 ± 0.26 | 1 | 1 |
| MS | 68.71 ± 0.41 | 4.61 ± 0.11 | 1.24 |
| MMS | 5.63 ± 0.06 | 0.38 ± 0.01 | 1.19 |
| C | 10.75 ± 0.21 | 0.72 ± 0.00 | 1.16 |

TABLE 9
S:MS = 1:4.31

| | | | before-and-after |
|---|---|---|---|
| | proportion | | ratio |
| | (%) | peak area ratio | A/B |
| <crude pigment> | | | |
| S | 15.35 ± 0.02 | 1 | — |
| MS | 66.22 ± 0.03 | 4.31 ± 0.00 | — |
| MMS | 6.20 ± 0.00 | 0.40 ± 0.00 | — |
| C | 12.23 ± 0.04 | 0.80 ± 0.00 | — |
| <composite> | | | |
| S | 11.94 ± 0.51 | 1 | 1 |
| MS | 70.42 ± 1.08 | 5.90 ± 0.25 | 1.37 |
| MMS | 5.23 ± 1.62 | 0.44 ± 0.17 | 1.1 |
| C | 12.37 ± 0.79 | 1.04 ± 0.03 | 1.3 |

As shown in the above Tables 4 to 9, the before-and-after ratio (A/B) of malonylshisonin (MS) was greater than the before-and-after ratio (1) of shisonin (S). For example, in Table 4 (S:MS=1:0.25), while the amount of MS is 0.25 times that of S in the crude pigment, the amount of MS increases to 2.08 times that of S in the composite. Similarly, in Table 5 (S:MS=1:0.4), while the amount of MS is 0.4 times that of S in the crude pigment, the amount of MS increases to 2.25 times that of S in the composite, and in Table 6 (S: MS=1:0.49), while the amount of MS is 0.49 times that of S in the crude pigment, the amount of MS increases to 2.20 times that of S in the composite. This demonstrates that malonylshisonin is incorporated more selectively than shisonin in the composite formation. From these results, it can be said that, when a composite is formed, the composite does not incorporate an arbitrary anthocyanin present in the vicinity thereof, but the incorporation of anthocyanins occurs with selectivity. More specifically, it was found that anthocyanins are incorporated in composites at different speeds depending on their structures, and comparing MS and S, MS is more selectively incorporated in the composite.

### Example 3

In Example 2, the selectivity of anthocyanins was confirmed. In Example 2, the crude pigment containing the respective kinds of anthocyanins and the Asiatic dayflower-derived flavocommelin were used in equal amounts (1:1 by weight ratio). When either the anthocyanins or the flavocommelin is present in excess, the difference in collision probability between the anthocyanins and the flavocommelin may affect the selectivity of the anthocyanins. Thus, the selectivity of the anthocyanins was checked under the condition where either the anthocyanins or the flavocommelin was present in excess.

Formation of composites and analysis of the components thereof by HPLC were carried out in the same manner as in Example 2, except that the amounts of a crude pigment and flavocommelin used were varied as follows: 3 mg of the crude pigment and 3 mg of the flavocommelin were used as the condition where anthocyanins and flavocommelin were present in equal amounts (1:1 by weight ratio); 2 mg of the crude pigment and 8 mg of the flavocommelin were used as the condition where the flavocommelin was present in excess; and 8 mg of the crude pigment and 2.7 mg of the flavocommelin were used as the condition where the anthocyanins were present in excess. Note here that the crude pigment and the flavocommelin used in the present example were the same as those in Example 1. These results are shown in the following tables.

TABLE 10
anthocyanin:flavocommelin = 1:1 (ratio by weight)

| | | | before-and-after |
|---|---|---|---|
| | proportion | | ratio |
| | (%) | peak area ratio | A/B |
| <crude pigment> | | | |
| S | 15.35 ± 0.02 | 1 | — |
| MS | 66.22 ± 0.03 | 4.31 ± 0.00 | — |
| MMS | 6.20 ± 0.00 | 0.40 ± 0.00 | — |
| C | 12.23 ± 0.04 | 0.80 ± 0.00 | — |
| <composite> | | | |
| S | 11.94 ± 0.51 | 1 | 1 |
| MS | 70.42 ± 1.08 | 5.90 ± 0.25 | 1.37 |
| MMS | 5.23 ± 1.62 | 0.44 ± 0.17 | 1.1 |
| C | 12.37 ± 0.79 | 1.04 ± 0.03 | 1.3 |

TABLE 11
anthocyanin:flavocommelin = 1:4 (ratio by weight)

| | | | before-and-after |
|---|---|---|---|
| | proportion | | ratio |
| | (%) | peak area ratio | A/B |
| <crude pigment> | | | |
| S | 15.35 ± 0.02 | 1 | — |
| MS | 66.22 ± 0.03 | 4.31 ± 0.00 | — |
| MMS | 6.20 ± 0.00 | 0.40 ± 0.00 | — |
| C | 12.23 ± 0.04 | 0.80 ± 0.00 | — |
| <composite> | | | |
| S | 13.71 ± 0.03 | 1 | 1 |
| MS | 63.42 ± 0.24 | 4.62 ± 0.02 | 1.07 |
| MMS | 9.89 ± 0.06 | 0.72 ± 0.00 | 1.8 |
| C | 13.03 ± 0.10 | 0.95 ± 0.01 | 1.12 |

TABLE 12
anthocyanin:flavocommelin = 2.96:1 (ratio by weight)

| | | | before-and-after |
|---|---|---|---|
| | proportion | | ratio |
| | (%) | peak area ratio | A/B |
| <crude pigment> | | | |
| S | 15.35 ± 0.02 | 1 | — |
| MS | 66.22 ± 0.03 | 4.31 ± 0.00 | — |
| MMS | 6.20 ± 0.00 | 0.40 ± 0.00 | — |
| C | 12.23 ± 0.04 | 0.80 ± 0.00 | — |
| <composite> | | | |
| S | 12.25 ± 0.01 | 1 | 1 |
| MS | 68.13 ± 0.03 | 5.56 ± 0.01 | 1.29 |
| MMS | 7.38 ± 0.01 | 0.60 ± 0.00 | 1.5 |
| C | 12.23 ± 0.02 | 1.00 ± 0.00 | 1.25 |

Under the flavocommelin excess condition (Table 11), the before-and-after ratios of malonylshisonin (MS) and cyanine (C) were 1.07 and 1.12, respectively. These values are smaller than the before-and-after ratios 1.37 and 1.3 of malonylshisonin (MS) and cyanine (C) under the equal amount condition (Table 10), which means the components changed only slightly before and after the composite formation. Conceivably, this is because the flavocommelin was present in excess relative to the anthocyanins under the flavocommelin excess condition as compared to the equal amount condition, so that the collision probability of the flavocommelin and the anthocyanins increased, thereby reducing the difference in selectivity of malonylshisonin (MS) and shisonin (C). On the other hand, under the anthocyanin excess condition (Table 12), the before-and-after ratio of malonylshisonin (MS) and cyanine (C) were 1.29 and 1.25, respectively. These values are equivalent to the before-and-after ratio 1.37 and 1.3 of malonylshisonin (MS) and cyanine (C) under the equal amount condition (Table 10), and no significant difference was observed. Although the amount of the anthocyanins was three times that of the flavocommelin under this condition, the obtained values were equivalent to those under the equal amount condition. From this, it was found that the amount of an anthocyanin does not affect the selectivity of the anthocyanin.

### Example 4

The four kinds of anthocyanins contained in the crude pigment were separated, and the selectivity in incorporation in a composite between arbitrary two components was checked.

### (1) Isolation of Anthocyanin

By medium pressure liquid chromatography, malonylshisonin (MS), shisonin (S), cyanine (C), and (methylmalonyl)shisonin (MMS) respectively were isolated from the crude pigment prepared in Example 1. First, in order to enhance the separation of the anthocyanins, 100 mL of a 100% stock solution (a mobile phase solution) shown below and 100 mL of a 20% stock solution (obtained by diluting the 100% stock solution with a 0.5% TFA aqueous solution, hereinafter the same) were caused to flow through the column in advance in this order at a flow rate of 2 mL/min for at least 30 minutes. 1 g of the crude pigment prepared in Example 1 was dissolved in a 20% stock solution, and the crude pigment was adsorbed to the tip of the glass column. The 20% stock solution was kept flowing through the glass column, and at the time point when the pigment had moved to the position at ⅔ of the glass column, the 20% stock solution was changed to a 30% stock solution (obtained by diluting the 100% stock solution with a 0.5% TFA aqueous solution). Thereafter, with the 30% stock solution flowing through the glass column, eluate was fractionated. Then, through visual observation, fractions containing the pigment at high concentrations were collected (200 mL/fraction). Subsequently, a 60% stock solution, an 80% stock solution, and a 100% stock solution as solvents were caused to flow through the column in this order, and fractionation of eluate and collection of fractions containing the pigment at high concentrations were carried out in the same manner as in the above. As a result, five fractions were collected with the 30% stock solution, three fractions were collected with the 60% stock solution, and one fraction was collected with the 80% stock solution, so that nine fractions were collected in total. By repeating the above-described procedure, 7 g of the crude pigment in total was obtained through fractionation and collection.

*MPLC conditions

| Fluid pump: | JASCO Corporation, trade name “PU-980” |
|---|---|
| Column: | trade name “ODS Develosil” |
| | 10 to 20 μm glass column with 300 × 25 mm i.d. |
| Mobile phase solution: | A 100% stock solution is composed of acetic |
| | acid:acetonitrile:water:trifluoroacetic acid = |
| | 20:2.5:54.5:0.5. |

As a result of fractionation and collection by MPLC, 72 mg of cyanine (Cy3,5-diglc), 76 mg of shisonin (Cy3-pc.glc-5-glc), 90 mg of malonylshisonin (Cy3-pc.glc-5-Ma.glc), and 44 mg of (methylmalonyl)shisonin (Cy3-pc.glc-5-Methoxy Ma glc) were obtained. The purity of malonylshisonin was 20%, and the purity of shisonin was 65%.

### *UV Spectrum Measurement Conditions

Device: JASCO Corporation,
trade name “V-520-SR type spectrophotometer”

Solvent: MeOH containing 0.1% HCl

Cell length: 1 mm

### *Absorption Spectrum Data of Malonylshisonin

UV-vis λ nm (ε): 529 (5640), 313.4 (3200), 292.5 (3580)

### *Absorption Spectrum Data of Shisonin

UV-vis λ nm (ε): 527 (13360), 313.4 (9560), 292.5 (10760)

### (2) Combination of Two Kinds of Anthocyanins

Two kinds of anthocyanins were mixed in the following combination so that the amounts of substances of these two components became equivalent. Thus, anthocyanin mixtures 1 and 2 were prepared.

Anthocyanin mixture 1: MS/MMS

Anthocyanin mixture 2: S/MMS

### (3) Analysis of Components of Composite

Formation of composites and analysis of the components thereof by HPLC were carried out in the same manner as in Example 2, except that two kinds of anthocyanin mixtures (1 and 2) were used instead of the crude pigment. The results are shown in the following tables.

TABLE 13
Anthocyanin mixture 1 (MS/MMS)

| | | before-and-after |
|---|---|---|
| | proportion | ratio |
| | (%) | A/B |
| <mixture> | | |
| MS | 37.45 ± 0.52 | — |
| MMS | 62.55 ± 0.52 | — |
| <composite> | | |
| MS | 52.51 ± 0.24 | 1.40 |
| MMS | 47.49 ± 0.24 | 0.76 |

TABLE 14
Anthocyanin mixture 2 (S/MMS)

| | | before-and-after |
|---|---|---|
| | proportion | ratio |
| | (%) | A/B |
| <mixture> | | |
| S | 46.20 ± 0.82 | — |
| MMS | 53.80 ± 0.82 | — |
| <composite> | | |
| S | 50.42 ± 0.06 | 1.10 |
| MMS | 49.58 ± 0.06 | 0.92 |

As shown in Table 13 above, in a system where the anthocyanin mixture 1 (MS/MMS) was used, the before-and-after ratio of MS was 1.4 times while the before-and-after ratio of MMS was 0.76 times. From this result, it can be said that MS has a higher selectivity than MMS (MS>>MMS). As shown in Table 14 above, in a system where the anthocyanin mixture 2 (SIMMS) was used, the before-and-after ratio of S was 1.10 times while the before-and-after ratio of MMS was 0.92 times. From this result, it can be said that S has a slightly higher selectivity than MMS (S>MMS).

The above results suggest that, with regard to the selectivity of the anthocyanins in the composite formation, there is no considerable difference between S and MMS, and MS has a particularly high selectivity (MS>>S≅MMS). Comparing malonylshisonin (MS) and shisonin (S), it is considered, from their structures, malonylshisonin is superior to shisonin in structural stability. Therefore, according to the results of the present example, it is presumed that, in the composite formation, there is a correlation between the stability of the composite and the selectivity of the anthocyanin.

### Example 5

Asiatic dayflower-derived commelinin occurring in nature is a metal complex composed of an anthocyanin (malonylawobanin), a flavone (flavocommelin), and magnesium (magnesium ion) present at a ratio of 6:6:2. Now, in the composite formation, the amount of magnesium relative to the anthocyanin was varied, and the influence thereof on the selectivity of the anthocyanin was checked.

Using malonylshisonin (MS) and shisonin (S) isolated in Example 4, an anthocyanin mixture shown below was prepared. Since MS and S were not purified substances with a purity of 100%, their ratios were indicated as an amount-of-substance ratio.

*Anthocyanin mixture

| | |
|---|---|
| composition | MS:S = 0.4:1 (amount-of-substance ratio) |
| | MS = 1 mg |
| | S = 1 mg |

Then, formation of composites was carried out in the same manner as in Example 1, except that: the above-described anthocyanin mixture was used instead of the crude pigment; the amounts of the anthocyanin mixture and the flavocommelin added (3 mg: 3 mg) were varied as shown below; and the amount of the magnesium acetate aqueous solution added was varied so as to be the following predetermined amounts.

### *Amount of Substance of Anthocyanin Mixture

1.1 μmol (S=1 mg, MS=1 mg)

### *Amount of Substance of Flavocommelin

1.1 μmol (1.5 mg)

### *Added Amount of 0.5 Magnesium Acetate Aqueous Solution

0.72 μL (0.36 μmol),
5 μL (2.5 μmol)
25 μL (12.5 μmol)
100 μL (50 μmol)
125 μL (62.5 μmol)

Then, with regard to the anthocyanin mixture and the formed composites, absorbance measurement was carried out in the same manner as in Example 1, and the yields (100×C/P) were determined. Furthermore, HPLC analysis of the composites was carried out in the same manner as in Example 2. The results are shown in the following table.

TABLE 15

| | amount-of- | | | | |
|---|---|---|---|---|---|
| | substance | | before- | | |
| | radio*¹ | peak | and- | | |
| | to | area | after ratio*³ | absorbance | yield |
| | anthocyanin | ratio*² | A/B | (abs) | (%) |
| <mixture> | | | | | |
| S | — | 1 | — | 0.603 | — |
| MS | — | 0.40 | — | (mixture) | |
| <composite> | | | | | |
| Mg²⁺ | | | | | |
| (μmol) | | | | | |
| 0.36 | 0.33 | 1.27 | 3.18 | 0.088 | 14.59 |
| 2.50 | 2.27 | 1.30 | 3.25 | 0.087 | 14.43 |
| 12.5 | 11.36 | 0.99 | 2.48 | 0.110 | 18.24 |
| 50.0 | 45.46 | 0.62 | 1.55 | 0.141 | 23.38 |
| 62.5 | 56.82 | 0.57 | 1.43 | 0.145 | 24.05 |

*¹The amount-of-substance ratio of Mg²⁺, determined by assuming that the amount of substance of the anthocyanin mixture was 1.
*²Assuming that a peak area (%) of shisonin in the anthocyanin mixture was 1, the ratio of a peak area of MS in the anthocyanin mixture and a peak area of MS in each composite is determined as a peak area ratio.
*³With regard to MS, the ratio of the peak area ratio (B) of MS in the anthocyanin mixture before the composite formation and the peak area ratio (A) of the composite (MS) after the composite formation was determined as a before-and-after ratio (A/B).

Furthermore, a graph plotting the molar ratio (amount-of-substance ratio) of Mg²⁺ to the mixed anthocyanins and the before-and-after ratio of malonylshisonin is shown in FIG. 3.

As shown in the above table and FIG. 3, under the condition where the magnesium was ⅓ equivalents relative to the anthocyanins (the anthocyanin mixture) (the amount-of-substance ratio to the anthocyanins was 0.33), MS increased 3.18 times (the before-and-after ratio A/B) from before to after the composite formation. Then, with the increase in the amount of the magnesium relative to the anthocyanins, the ratio (A/B) of MS before and after the composite formation decreased. Specifically, under the condition where the magnesium was 56.82 equivalents relative to the anthocyanins (the amount-of-substance ratio to the anthocyanins was 56.82), the increase in MS from before to after the composite formation decreased to 1.43 times. From this result, it was found that, as the amount of substance of the magnesium relative to the anthocyanins became smaller, the increase rate of the composite incorporating MS became higher, and as the amount of substance of the magnesium relative to the anthocyanins became larger, the increase rate of the composite incorporating MS became lower. Therefore, by adjusting the amount of magnesium in the composite formation, it becomes possible to control the composition of shisonin and malonylshisonin, for example. This brings about a further advantageous effect that, in anthocyanin purification, perilla-derived anthocyanins contained in a composite can be extracted selectively. In particular, this is useful when selectively isolating shisonin rather than malonylshisonin since a malonyl group is unstable toward acids.

### Example 6

In the composite formation, the amount of flavocommelin relative to anthocyanins was varied, and the influence thereof on the selectivity of the anthocyanins was checked. The amount of magnesium ion was set to be an excess amount.

The formation of a composite was carried out in the same manner as in Example 1, except that the condition used in Example 1 were changed to the following conditions.

TABLE 16
<Condition 1>
*Anthocyanin mixture

| | |
|---|---|
| composition | MS:S = 0.4:1 (amount-of-substance ratio) |
| | MS = 1 mg |
| | S = 1 mg |
| amount of substance | 1.1 μmol |

*Flavocommelin

| | |
|---|---|
| added amount | 0.75 mg (1.12 μmol) |
| | 1.5 mg (2.23 μmol) |
| | 3.0 mg (4.46 μmol) |
| | 7.5 mg (11.2 μmol) |

*0.5 magnesium acetate aqueous solution

| | |
|---|---|
| added amount | 100 μL (50 μmol) |

<Condition 2>
*Anthocyanin mixture

| | |
|---|---|
| composition | MS:S = 5.44:1 (amount-of-substance ratio) |
| | MS = 0.1 mg |
| | S = 3 mg |
| amount of substance | 0.83 μmol |

*flavocommelin

| | |
|---|---|
| added amount | 0.56 mg (0.83 μmol) |
| | 1.12 mg (1.65 μmol) |
| | 2.24 mg (3.30 μmol) |
| | 5.6 mg (8.25 μmol) |

*0.5 magnesium acetate aqueous solution

| | |
|---|---|
| added amount | 100 μL (50 μmol) |

Then, with regard to the anthocyanin mixtures and the formed composites, the absorbance measurement was conducted in the same manner as in Example 1 and the yields (100×C/P) were determined. Furthermore, HPLC analysis of the composites was carried out in the same manner as in Example 2. The results are shown in Tables 17 and 18 below. Table 17 shows the result concerning the composites of the condition 1, and Table 18 shows the result concerning the composite of the condition 2.

TABLE 17
<Condition 1> MS:S = 0.40:1

| | amount-of- | | | | |
|---|---|---|---|---|---|
| | substance | | | | |
| | ratio*¹ | peak | before-and- | absorb- | |
| | to | area | after ratio*³ | ance | yield |
| | anthocyanin | ratio*² | A/B | (abs) | (%) |
| <mixture> | | | | | |
| S | — | 1 | — | 0.603 | — |
| MS | — | 0.40 | — | (mixture) | |
| <composite> | | | | | |
| F*⁴ (μmol) | | | | | |
| 1.12 | 1.02 | 0.56 | 1.40 | 0.176 | 29.19 |
| 2.23 | 2.03 | 0.62 | 1.55 | 0.141 | 23.38 |
| 4.46 | 4.05 | 0.64 | 1.60 | 0.259 | 42.95 |
| 11.2 | 10.18 | 0.52 | 1.30 | 0.261 | 43.28 |

*¹The amount-of-substance ratio of flavocommelin, determined by assuming that the amount of substance of the anthocyanin mixture was 1.
*²Assuming that a peak area (%) of shisonin in the anthocyanin mixture was 1, the ratio of a peak area of MS in the anthocyanin mixture and a peak area of MS in each composite is determined as a peak area ratio.
*³With regard to MS, the ratio of the peak area ratio (B) of MS in the anthocyanin mixture before the composite formation and the peak area ratio (A) of the composite (MS) after the composite formation was determined as a before-and-after ratio (A/B).
*⁴flavocommelin

TABLE 18
<Condition 2> MS:S = 5.44:1

| | amount-of- | | | | |
|---|---|---|---|---|---|
| | substance | | | | |
| | ratio*¹ | peak | before-and- | absorb- | |
| | to | area | after ratio*³ | ance | yield |
| | anthocyanin | ratio*² | A/B | (abs) | (%) |
| <mixture> | | | | | |
| S | — | 1 | — | 0.535 | — |
| MS | — | 5.44 | — | (mixture) | |
| <composite> | | | | | |
| F*⁴ (μmol) | | | | | |
| 0.83 | 1.00 | 5.82 | 1.08 | 0.292 | 54.58 |
| 1.65 | 2.00 | 5.36 | 0.98 | 0.237 | 44.30 |
| 3.30 | 4.00 | 5.59 | 1.03 | 0.295 | 55.14 |
| 8.25 | 10.00 | 5.53 | 1.02 | 0.3 | 57.01 |

*¹The amount-of-substance ratio of flavocommelin, determined by assuming that the amount of substance of the anthocyanin mixture was 1.
*²Assuming that a peak area (%) of shisonin in the anthocyanin mixture was 1, the ratio of a peak area of MS in the anthocyanin mixture and a peak area of MS in each composite is determined as a peak area ratio.
*³With regard to MS, the ratio of the peak area ratio (B) of MS in the anthocyanin mixture before the composite formation and the peak area ratio (A) of the composite (MS) after the composite formation was determined as a before-and-after ratio (A/B).
*⁴flavocommelin

As shown in Table 17 above, when the anthocyanin mixture (MS:S=0.40:1) was used and the amount of the flavocommelin was varied, the increase rate (A/B) of MS from before to after the composite formation was around 1.5 regardless of the amount of the flavocommelin. Furthermore, as shown in Table 18 above, when the anthocyanin mixture (MS:S=5.44:1) was used and the amount of the flavocommelin was varied, the increase rate (A/B) of MS from before to after the composite formation was about 1 time, which means little change in composition was observed regardless of the amount of flavocommelin. These results demonstrate that, under the magnesium excess condition, the amount of flavocommelin does not particularly affect the selectivity of anthocyanins.

The present example employed a magnesium excess condition where 45 equivalents of magnesium was used relative to the anthocyanin mixture, and further, an excess amount of flavocommelin, namely 1 to 10 equivalents relative to the anthocyanins, was used. Therefore, theoretically, it was a condition where all the anthocyanins could be used for the composite formation. However, in the condition 1 (MS: S=0.40:1), not all of the anthocyanins were incorporated, and as described above, the compositional ratio of MS increased around 1.5 times after the composite formation. On the other hand, in the condition 2 (MS:S=5.44:1), there was little change in compositional ratio of MS between the anthocyanin mixture and the composite. Furthermore, in the condition 1 (MS: S=0.40:1), the maximum yield of the composite was 57.01%, while in the condition 2 (MS:S=5.44:1), the maximum yield of the composite was 43.28%, which is about 14% lower than that in the condition 1. From these results, it is considered that, in the composite formation, even when a crude pigment raw material (anthocyanin mixture) in which the compositional ratio of MS is low as in the condition 1 is used, MS having a higher selectivity than S is incorporated prior to S, so that the ratio of MS increases after the composite formation, while, in contrast, the yield of the composite decreases.

### Example 7

In Examples 1 to 6, flavocommelin was used. In the following, the formation of composites was checked using other flavones.

First, a TFA salt of a perilla crude pigment was provided. The TFA salt of the perilla crude pigment was obtained by drying out a liquid pigment extract of red perilla leaves, extracted by the method described in the item (1) of Example 1. The TFA salt was not subjected to the purification by column chromatography. On the other hand, as a flavone, apigenin 7-glucoside (Funakoshi Co., Ltd.) was used.

Next, about 9 mg of the TFA salt of the perilla crude pigment was dissolved in 150 μl of ultrapure water. To this solution, 1 mL of 0.5 M ammonia aqueous solution was added, and the resultant mixture was concentrated to dryness under reduced pressure immediately. 10 μmol of the apigenin 7-glucoside was measured out and added thereto while remaining in the solid state. Then, 600 μl (300 μmol) of a 50% ethanol aqueous solution containing 0.5 M magnesium acetate was further added thereto. This was dissolved by applying ultrasonic vibrator as appropriate. The resultant solution was then concentrated to dryness and cryopreserved until the analysis. Next, the cryopreserved sample was dissolved in a minimum amount of ultrapure water and adsorbed to Sephadex G-10 (trade name) gel filtration column chromatography (0.8 to 1.0 cm×10.5 to 11.5 cm). Ultrapure water then was caused to flow through the column. Thereafter, two fractions, which were eluted earliest in the Sephadex G-10 and had the highest pigment concentration, were collected. As a result, in the fractions eluted earliest in the Shephadex, there was a fraction in which a pigment and apigenin 7-glucoside were eluted. This indicates that the pigment and this flavone coprecipitated by the addition of ethanol. That is, it was confirmed that perilla-derived anthocyanins can be purified with apigenin 7-glucoside.

Furthermore, the same operation as in Example 7 was carried out except that, instead of apigenin 7-glucoside, each of the following components was used: flavocommelin; hesperidin and diosmin, which are citrus fruit-derived flavones; naringenin 7-glucoside, which is a citrus fruit-derived flavone; rutin, which is a buckwheat-derived flavone; apigenin; apigenin 7-o-neohesperidoside; silymarin; and luteolin 7-glucoside, which is an olive-derived flavone (all available from Funakoshi Co., Ltd.). As a result, coprecipitation of each of the flavones with the perilla-derived anthocyanins was confirmed.

### INDUSTRIAL APPLICABILITY

According to the method for producing a purified anthocyanin of the present invention, it is possible to purify an anthocyanin from a crude pigment fraction easily in a simple manner. Moreover, it becomes possible to provide a purified anthocyanin at low cost. Hence, it can be said that the present invention is very useful in all the fields where natural pigments are used, including, for example, a food field.

1. A method for producing a purified anthocyanin from a crude pigment fraction containing the anthocyanin, comprising the steps of
(A) providing a flavonoid that forms a metal complex with the anthocyanin and a metal ion of at least one of an alkaline-earth metal and a heavy metal, the flavonoid being a flavonoid derived from a plant;
(B) bringing the flavonoid into contact with the crude pigment fraction in a liquid in the presence of the metal ion of at least one of the alkaline-earth metal and the heavy metal, thereby forming a metal complex containing the anthocyanin, the flavonoid, and the metal ion;
(C) collecting the metal complex from the liquid; and
(D) dissociating the anthocyanin from the metal complex.

2. The method according to claim 1, wherein the flavonoid is at least one flavone selected from the group consisting of flavones derived from Asiatic dayflower (Commelina communis), flavones derived from cornflower (Centaurea cyanus), flavonols derived from Himalayan blue poppy (Meconopsis hetonicifolia), and flavones derived from blue salvia (Salvia farinncea).

3. The method according to claim 1, wherein the flavonoid is at least one selected from the group consisting of flavocommelin, apigenin 7,4′-diglucoside, apigenin 4′-(6-O-malonylglucoside)-7-glucuronide, flavonol 3-getiobiose, and flavonol 3-(6-O-glucosyl-b-O-galactoside).

4. The method according to claim 1, wherein the crude pigment contains two or more kinds of anthocyanins.

5. The method according to claim 1, wherein the anthocyanin is at least one anthocyanin selected from the group consisting of anthocyanins as peonidin-based glycosides, anthocyanins as delphinidin-based glycosides, anthocyanins as petunidin-based glycosides, and anthocyanins as delphinidin-based glycosides, each having at least two OH groups in a B-ring.

6. The method according to claim 1, wherein the crude pigment fraction is a crude pigment fraction containing an anthocyanin extracted from a plant, the plant being at least one plant selected from the group consisting of perillas, red cabbages, grapes, black corns, red radishes, berries, beans, potatoes, rice, red onions, olives, and apples.

7. The method according to claim 1, wherein the alkaline-earth metal ion is a magnesium ion.

8. The method according to claim 1, wherein the heavy metal ion is at least one metal ion selected from the group consisting of zinc, nickel, cadmium, iron, cobalt, aluminum, copper, manganese, chromium, and tin.

9. The method according to claim 1, further comprising, prior to the step (B), the step of treating the crude pigment fraction with alkali to transform the anthocyanin contained in the crude pigment fraction into an anhydro base or an anhydro base anion.

10. The method according to claim 1, wherein, in the step (C), the metal complex is collected from the liquid by ethanol precipitation or molecular weight fractionation.

11. The method according to claim 10, wherein, in the step (C), the molecular weight fractionation is gel filtration.

12. The method according to claim 1, wherein, in the step (D), the anthocyanin is dissociated from the metal complex by subjecting the metal complex to at least one treatment selected from the group consisting of an acid treatment, a heat treatment, and an ultrasonic treatment.

13. The method according to claim 1, wherein
in the step (D), the anthocyanin is dissociated from the metal complex by subjecting the metal complex to an acid treatment,
after the step (D), a mixture containing the anthocyanin that has been positively charged by the acid treatment in the step (D), the flavonoid that is electrically neutral, and the metal ion is applied to a cation-exchange resin, whereby the anthocyanin is adsorbed to the cation-exchange resin, and
after removing the flavonoid, the adsorbed anthocyanin is eluted.

14. A purified anthocyanin obtained by the production method according to claim 1.
